(19) United States
(12) Patent Application Publication
(10) Pub. No.: US 2015/0141962 A1
(43) Pub. Date: May 21, 2015

(54) SECURING DEVICE FOR MEDICAL LINES

(71) Applicants: Steve A. Collins, Blue Ridge, GA (US); Cynthia G. Collins, Blue Ridge, GA (US)

(72) Inventors: Steve A. Collins, Blue Ridge, GA (US); Cynthia G. Collins, Blue Ridge, GA (US)

(21) Appl. No.: 14/606,404

(22) Filed: Jan. 27, 2015

Related U.S. Application Data
(63) Continuation-in-part of application No. 14/061,834, filed on Oct. 24, 2013.
(60) Provisional application No. 61/932,299, filed on Jan. 28, 2014.

(51) Int. Cl. A61M 25/02; A61M 5/158

(52) U.S. Cl. CPC A61M 25/02; A61M 5/158; A61M 2005/1586; A61M 2025/0206; A61M 2025/024; A61M 2025/0253
USPC 604/513; 604/180

(57) ABSTRACT

Disclosed are various embodiments for devices configured to secure medical lines. A securing frame is configured to receive and secure at least one line couplings. The at least one line coupling is be configured to at least partially surround an intravenous such that the intravenous line is secured in an aperture of the line coupling. The line couplings can be detachably attached to a portion of the securing frame, for example, between concave portions of an upper arm and a lower arm, wherein the upper arm and the lower arm together comprise a latching mechanism. As a result, the securing frame can be latched to further secure the at least one intravenous line. The stability and control provided by the line securing device prevents movement of the intravenous line and/or needle relative to an insertion point on a patient, thereby preventing patient injury.

### CROSS-REFERENCE TO RELATED APPLICATIONS

This application is a continuation-in-part of and claims priority to U.S. patent application Ser. No. 14/061,834 entitled “SECURING DEVICE FOR MEDICAL LINES,” filed on Oct. 24, 2013, which is incorporated herein by reference in its entirety. This application further claims priority to U.S. Provisional Patent Application No. 61/932,299 entitled “SECURING DEVICE FOR MEDICAL LINES,” filed on Jan. 28, 2014, which is incorporated herein by reference in its entirety.

### BACKGROUND

Intravenous line and catheter complications can occur if an intravenous line is positioned incorrectly or becomes dislodged from a vein. For instance, if pressure is not applied firmly when an intravenous line is removed, blood can leak out of the vein and build up in the tissues resulting in pain, swelling, infection, and blood clots. The complications associated with intravenous therapy can result in not only a rise in healthcare costs from prolonged hospitalization and an extended use of antibiotic therapy, but also surgical intervention and possibly even death. Intravenous therapy complications happen regularly as a result of patients accidentally dislodging an intravenous line or catheter from the vein. Thus, these lines and catheters need to be secured to the patient to avoid such accidental removal from the vein.

### BRIEF DESCRIPTION OF THE DRAWINGS

Many aspects of the present disclosure can be better understood with reference to the following drawings. The components in the drawings are not necessarily to scale, with emphasis instead being placed upon clearly illustrating the principles of the disclosure. Moreover, in the drawings, like reference numerals designate corresponding parts throughout several views.

FIG. 1 is a drawing of a substance delivery system according to various embodiments of the present disclosure.

FIG. 2A is a drawing of one embodiment of an access device.

FIG. 2B is a drawing of another embodiment of an access device.

FIG. 3 is a drawing of an embodiment of an intravenous extension tube.

FIGS. 4A-4B are drawings of adhesive backings for the access device of FIG. 2A comprising releasable locking devices.

FIGS. 5A-5B are drawings of adhesive backings for the access device of FIG. 2B comprising releasable locking devices.

FIGS. 6A-6B are drawings illustrating an embodiment of an intravenous line securing system.

FIGS. 7A-7B are drawings illustrating an embodiment of a multi-port line securing system.

FIGS. 8A-8C are drawings showing perspective views of one embodiment of the releasable locking device.

FIGS. 9A-9H are drawings showing alternative embodiments of the releasable locking device.

FIGS. 10A-B are drawings showing alternative embodiments of the intravenous line securing system depicted in FIGS. 6A-6B.

FIG. 11 is a flowchart illustrating one example of functionality implemented by a user of a substance delivery system comprising the line securing system according to various embodiments of the present disclosure.

FIGS. 12A-C are drawings showing perspective views of various embodiments of an adhesive clamp.

FIG. 13 is a drawing of an embodiment of an access device with a hub located on a catheter tube of the access device.

FIG. 14 is a drawing of an embodiment of an intravenous line securing system comprising a frame and a line coupling according to various embodiments of the present disclosure.

FIGS. 15A-B are drawings showing perspective views of the embodiment of the securing frame of the intravenous line securing system of FIG. 14 according to various embodiments of the present disclosure.

FIGS. 16A-C are perspective drawings of a line coupling of FIG. 14 according to various embodiments of the present disclosure.

FIGS. 17A-D are perspective drawings of another embodiment of an intravenous line securing system according to various embodiments of the present disclosure.

FIGS. 18A-D are perspective drawings of yet another embodiment of an intravenous line securing system according to various embodiments of the present disclosure.

FIG. 19 is a flowchart illustrating functionality implemented by a user of a substance delivery system comprising a line securing system according to various embodiments of the present disclosure.

### DETAILED DESCRIPTION

The present disclosure relates to securing intravenous lines and central venous lines. In some embodiments, the systems include an intravenous extension tube with a specially designed attachable portion. One end of the intravenous extension tube is affixed to a source intravenous tubing that receives a substance from a solution delivery bag. The other end of the intravenous extension tube is affixed to a target intravenous tubing. The intravenous extension tube can be configured to attach to a releasable locking device of an adhesive backing attachable to a patient. A solution, or other content, can travel from the solution delivery bag, through the source intravenous tubing, through the intravenous extension tube, and through the target intravenous tubing into an access device that injects the solution into the patient.

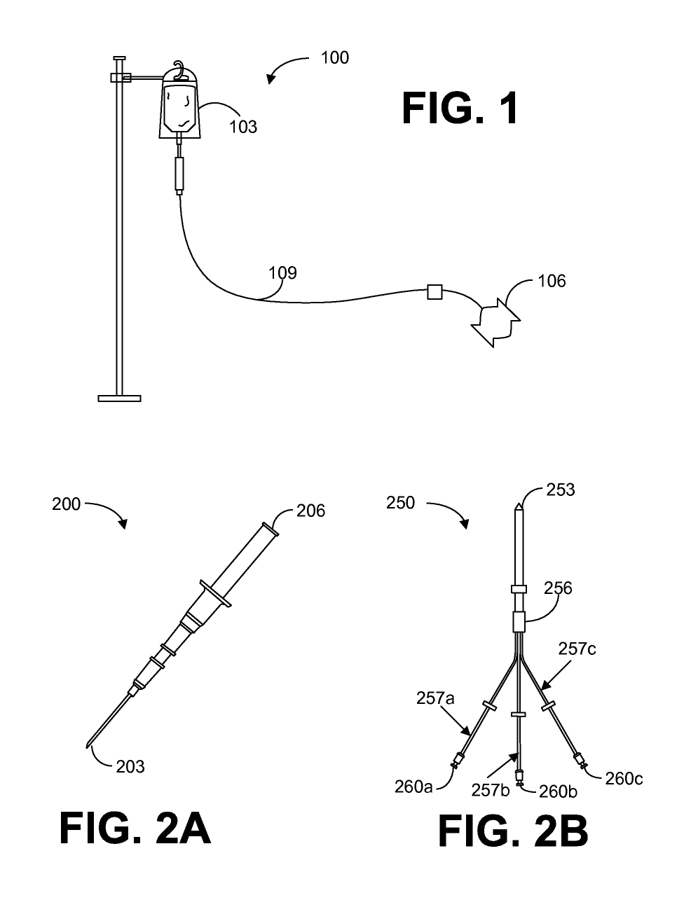

FIG. 1 is a drawing of a substance delivery system according to various embodiments of the present disclosure. The substance delivery system 100 comprises at least one substance delivery bag 103 connected to an adhesive backing 106 by means of one or more lines 109. The lines 109 can be intravenous tubing, electrical lines, drainage tubes, or lumen tubing. The substance delivery bag 103 can comprise one or more pre-formed concentrated solutions that can be diluted for intravenous injection. For example, the substance delivery bag 103 can contain fluids containing electrolytes to be delivered through the line 109 to a dehydrated patient. The solutions from the substance delivery bag 103 are to be injected into the vein of a patient at an injection site. Traditionally, the line 109 can be stabilized at the injection site by using sutures, tunnelers, wings, anchor pads, clamps, injection caps, and/or any Statlock® stabilization device. The line 109 can combine solutions from the substance delivery bag 103. At least a portion of the line 109 can be coupled to the adhesive backing 106. The adhesive backing 106 can also be partially attached to an infusion member configured to inject the solutions into the patient at the injection site. As can be appreciated, the injection sites include, but are not limited to, the hand, arm, neck, or leg of a patient. A patient can be a human, an animal, any living being, or an electrical device, including but not limited to, a robot, vehicle, machine, or computer. The line 109 can be an electrical cable or wire, and the injection site can be a location of the electrical device, including, but not limited to, a robot, vehicle, machine, or computer. As can be appreciated, the solution delivery bag mentioned throughout this disclosure can also represent a central line hub.

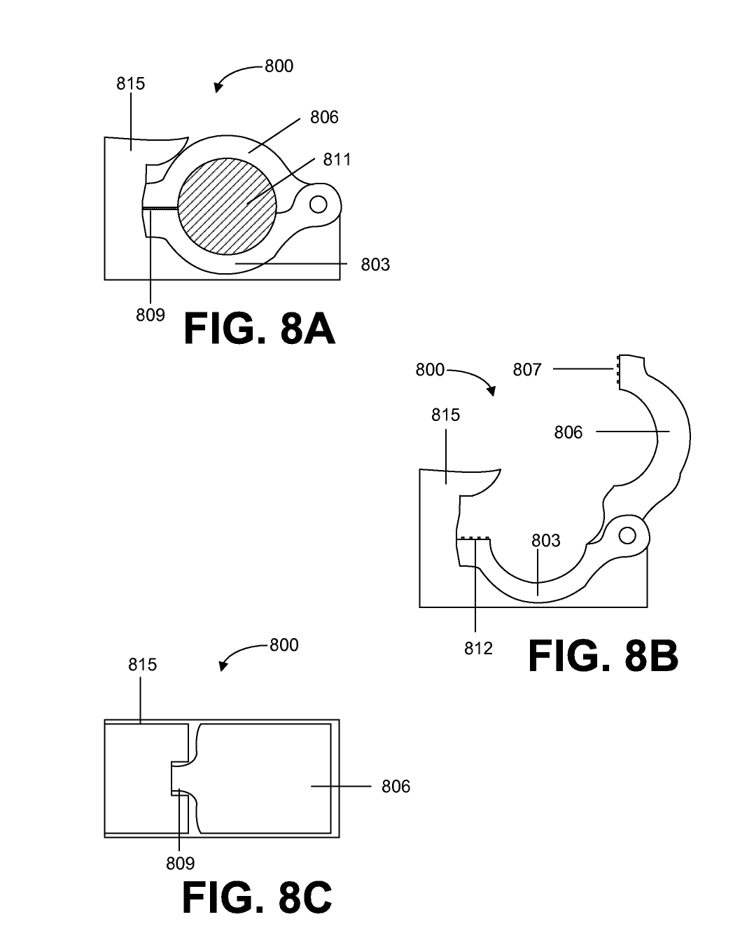

In one embodiment of the present disclosure, an intravenous extension tube can be disposed on an end of the line 109, and the intravenous extension tube can be attached to a releasable locking device on the adhesive backing 106. Examples of such a releasable locking device are further described below in relation to FIGS. 8A-C and 9A-F. In such an embodiment, the solutions can travel from the substance delivery bag 103, through the line 109, through the intravenous extension tube, through the access device, and into the patient.

In another embodiment, the intravenous extension tube can be displaced in between a first portion of the line 109 and a second portion of the line 109. The intravenous extension tube can be coupled to a releasable locking device of the adhesive backing 106. The first portion of the line 109 can channel the solution from the substance delivery bag 103. The second portion of the line 109 can be coupled to an access device or an infusion member configured to inject the solutions into the patient at the injection site.

FIG. 2A is a drawing of one embodiment of an access device 200. As can be appreciated, an access device 200 can be, but is not limited to, a drainage device, an imaging device, an epidural line, a dialysis catheter, a neonatal line, a pediatric line, an intravenous line, or any line or device that can be inserted directly into a patient's body. A drainage device can be a surgical drain tube used to remove pus, blood, or other fluids from a wound. As such, a drainage device can be inserted anywhere into a patient's body where there is a need for such drainage. Examples of drain tubes include, but are not limited to, a nephrostomy tube, a thoracic tube, a Jackson-Pratt® drain, a Penrose drain, a negative pressure wound therapy drain, a redivac drain, a pigtail drain, a davol, a wound managing drain, or any drainage tube that can be inserted into the patient's body.

For example, a patient suffering from internal hydrocephalus, also known as water on the brain, has an accumulation of fluids in the ventricles of the brain. The internal hydrocephalus can be successfully treated by placing a drainage tube between the brain ventricles and abdominal cavity to eliminate the high intracranial pressure. In such a case, the patient can suffer from fatal injury if the drainage tube is displaced at any time during the drainage. As such, the drainage tube can be secured by using the securing device for medical lines disclosed herein.

As another example, a patient with kidney failure can be treated using a nephrostomy tube that allows for the urinary diversion directly from the upper part of the urinary system. The nephrostomy tube can be inserted into the patient at a nephrostomy insertion site created between the kidney and the skin. Similar to the example above, the patient can suffer from infection or other serious harm if the nephrostomy tube is dislodged from the nephrostomy insertion site. The nephrostomy tube can be secured using the securing device for medical lines disclosed herein. Similarly, any patient receiving treatment using a drainage tube can utilize the securing device to secure the drainage tube to the patient at the insertion site to prevent injury from dislodging of the drainage tube.

An intravenous line, as illustrated in FIG. 2A, can be, but is not limited to, an arterial, central, midline, or peripheral intravenous line. The intravenous line access device 200 can comprise, for example, a needle 203, a tube inlet 206, and other components. The needle 203 can be configured to inject into a vein of a patient. In one embodiment, the tube inlet 206 is coupled to the intravenous extension tube. In such an embodiment, the tube inlet 206 is configured to receive solutions directly from the intravenous extension tube and then inject the solutions into the patient. In another embodiment, the tube inlet 206 can be coupled to the second portion of a line 109 (FIG. 1). Similar to the embodiment discussed above in relation to FIG. 1, the solutions can travel through the intravenous extension tube through the second portion of the line 109 before traveling through the tube inlet 206 and the needle 203.

FIG. 2B is a drawing of another embodiment of an access device. More specifically, FIG. 2B is a drawing of a multi-port indwelling catheter 250. A multi-port indwelling catheter 250 can be a central venous catheter. Such a central venous catheter can be a catheter placed into a large vein in the neck (internal jugular vein), chest (subclavian vein or axillary vein), or groin (femoral vein). The central venous catheter can be used to administer medication or solutions, obtain blood tests, and directly obtain cardiovascular measures such as, but not limited to, the central venous pressure. One embodiment of a central venous catheter can use a non-tunneled catheter that is fixed in a place at the insertion site, with the catheter and attachments protruding directly. Non-tunneled catheters can include Quinton catheters.

Another embodiment of a central venous catheter can use a tunneled catheter. Tunneled catheters are passed under the skin from the insertion site to a separate exit site, where the catheter and its attachments emerge from underneath the skin. The exit site is typically located in the chest, making the access ports less visible than if they were protruding directly from the neck. Passing the catheter under the skin helps to prevent infection and provides stability. Tunneled catheters can include Hickman catheters and Groshong catheters.

Another embodiment of a central venous catheter can use an implanted port. A port is similar to a tunneled catheter but is left entirely under the skin. Medicines are injected through the skin into the catheter. Some implanted ports contain a small reservoir that can be refilled in the same way. After being filled, the reservoir slowly releases the medicine into the bloodstream. Yet another embodiment of a central venous catheter is a peripherally inserted central catheter line, which is a central venous catheter inserted into a vein in the arm rather than a vein in the neck or chest.

FIG. 2B illustrates a multi-port indwelling catheter 250 that can couple to three lines. The multi-port indwelling catheter 250 can comprise a central needle 253, a catheter tube 256, intermediaries 257a-c, and line ports 260a-c. Each of the intermediaries can represent an intravenous tube, an electrical line, and/or a single or multi lumen tubing that is displaced between an intravenous extension tube and the catheter tube 256. Each of the line ports can represent, for example, a proximal, medial, or distal. Each of the line ports can be coupled to a separate intravenous tubing or electric line.

The central needle 253 can be inserted into the patient at an injection site. The line ports 260a-c can attach to a respective intravenous extension tube. Each intravenous extension tube can comprise an attachable portion configured to attach to a releasable locking device of an adhesive backing secured to a patient. In one embodiment, each intravenous extension tube can be connected to the respective line port 260a-c at one end and the catheter tube 256 at another end of the intravenous extension tube. In an alternative embodiment, one intravenous extension tube can be disposed at the catheter tube 256. In yet another embodiment, one end of the intravenous extension tube can be connect to the respective line port 260a-c and the other end of the intravenous extension tube can be connected to lines connected to or channel fluids from at least a portion of a central hub.

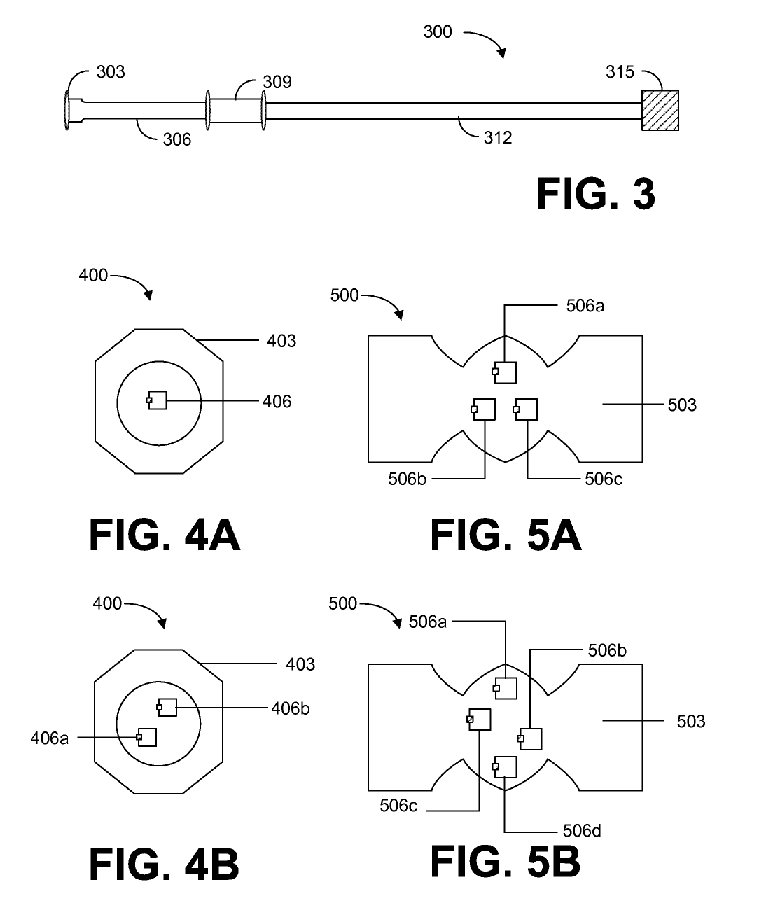

FIG. 3 is a drawing of an embodiment of an intravenous extension tube 300. The intravenous extension tube 300 comprises an inlet connector 303, a first section 306, an attachable portion 309, a second section 312, and an outlet connector 315. The inlet connector 303 can couple to a source intravenous line that channels solutions to be delivered to the patient. Alternatively the inlet connector 303 can couple to an electric cable configured to monitor equipment of the solution delivery system and/or obtain cardiovascular measures of a patient. The inlet connector 303 can couple to a first section 306 of the intravenous extension tube 300. The first section 306 of the intravenous extension tube 300 can couple to the attachable portion 309. The attachable portion 309 can be configured to attach to a releasable locking device of an adhesive backing to be secured to a patient. The attachable portion 309 can be coupled to the second section 312 of the intravenous extension tube 300. In one embodiment, the second section 312 can be made of a more rigid material than the first section 306. In another embodiment, the first section 306 can be made of a more rigid material than the second section 312. In yet another embodiment, the first section 306 can be of the same rigidness of the second section 312. The second section 312 can be coupled to an outlet connector 315. The outlet connector 315 can be configured to couple to a portion of the intravenous tubing or medical line. Alternatively, the outlet connector 315 can couple directly to the access device. According to the various embodiments, the intravenous extension tube 300 can be about eight inches long.

As an illustrative example, the intravenous extension tube 300 can be used in connection with the access device described in FIG. 2A. The inlet connector 303 can connect to a line of a substance delivery system. In one embodiment, the outlet connector 315 can connect to the tube inlet 206 of FIG. 2A. In another embodiment, the outlet connector 315 can connect anywhere on the access device 200 of FIG. 2A. The outlet connector 315 can alternatively connect to sutures, tunnelers, wings, anchor pads, clamps, injection caps, and/or any Statlock® stabilization device used on or in proximity to the insertion site. The intravenous extension tube 300 can serve as an intermediary between the line and the access device 200 represented in FIG. 2A.

As another illustrative example, a plurality of intravenous extension tubes 300 can be used in connection with the multi-port indwelling catheter 250 described in FIG. 2B. Each of the inlet connectors 303 can respectively connect to a line of a substance delivery system. In one embodiment, each of the outlet connectors 315 can be coupled to one of the line ports 260a-c of FIG. 2B. In another embodiment, the outlet connector 315 can be coupled to the catheter tube 256 of FIG. 2B. The intravenous extension tube 300 can serve as an intermediary between the needle 253 (FIG. 2B) and the line 109 (FIG. 1) to be injected into the patient and each of the lines.

As an example of the functionality of the intravenous extension tube 300, a patient receiving fluids from a substance delivery system, as described in FIG. 1, can have the access device 200 or multi-port indwelling catheter 250 from FIG. 2A or FIG. 2B secured to the patient's body using an adhesive bandage comprising a releasable locking device. The intravenous extension tube 300 can be located in between the access device and the line 109 from FIG. 1. Suppose a patient accidentally knocks down the line 109 from FIG. 1, then the releasable locking device will detach from the intravenous extension tube 300, preventing the needle 253 (FIG. 2B) of the multi-port indwelling catheter 250 (FIG. 2B) from dislodging inside the patient.

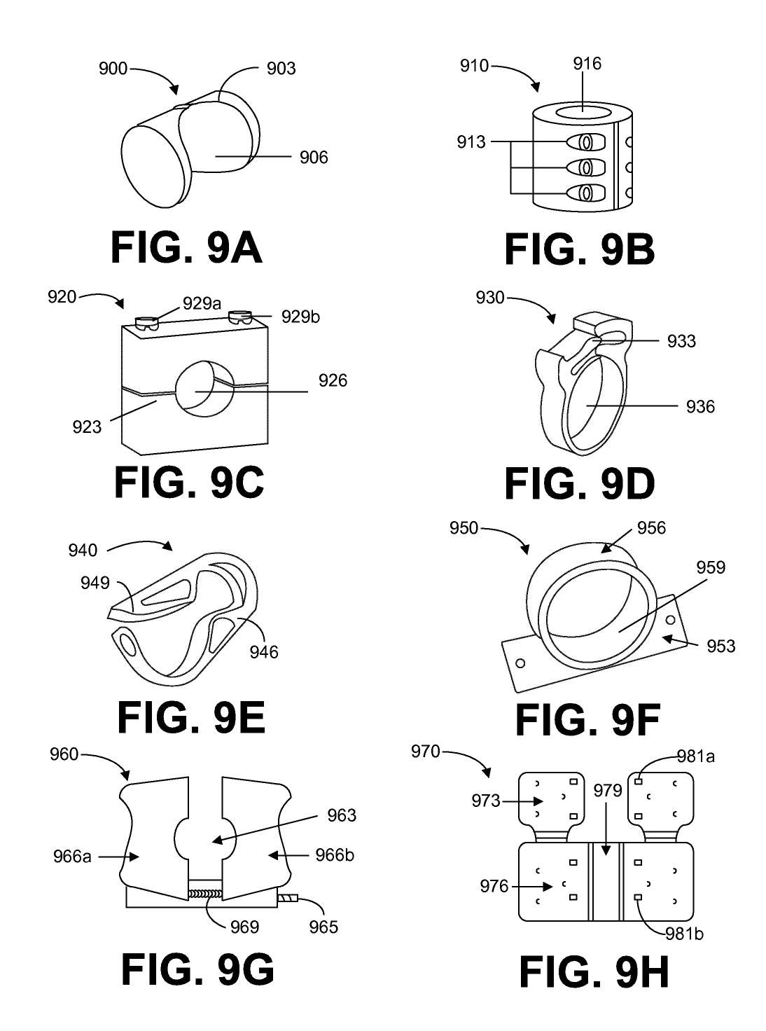

FIGS. 4A-4B are drawings of adhesive backings for intravenous lines that comprise releasable locking devices. FIG. 4A illustrates an adhesive backing 400 comprising an adhesive portion 403 and one releasable locking device 406. In one embodiment, the adhesive backing 400 can be configured to be secured to the patient in proximity to an injection site using the adhesive portion 403. As can be appreciated, the adhesive backing can sterile or non-sterile. The adhesive backing can also be transparent and further comprise a gauze pad. The releasable locking device 406 can be configured to detachably attach to the attachable portion of an intravenous extension tube, as described above in FIG. 3 in relation to the attachable portion 309. Examples of structures of the releasable locking device 406 that can be employed in the non-limiting example of FIG. 4A are described further in FIGS. 8 and 9. As can be appreciated, the adhesive backing 400 can be made of any material that can be attached together tightly around patient at a location of the patient's body.

FIG. 4B illustrates the adhesive backing 400 comprising the adhesive portion 403 and two releasable locking devices 406a-b. Each of the releasable locking devices 406a-b can be configured to attach to an intravenous extension tube of a two-port indwelling catheter line similar to the releasable locking device 406 described in FIG. 4A. As illustrated in FIG. 4B, the releasable locking devices 406a-b can be positioned on the adhesive backing in a staggered manner such that an intravenous extension tube 300 (FIG. 3) can pass through each one of the releasable locking device 406a-b. Alternatively, the releasable locking devices 406a-b can be positioned on the adhesive backing in a matrix form, where each of the releasable locking devices 406a-b are positioned parallel and perpendicular to one another.

FIGS. 5A-5B are drawings of adhesive backings for multi-port indwelling catheters comprising a plurality of releasable locking devices. FIG. 5A illustrates an adhesive backing 500 comprising an adhesive portion 503 and three releasable locking devices 506a-c. The three releasable locking devices 506a-c can be configured to detachably attach to the attachable portions of a trilumen indwelling catheter, similar to the one shown in FIG. 2B. The releasable locking devices 506a-c of the adhesive backing 500 can operate similar to the releasable locking device 406 described above in relation to FIG. 4A.

FIG. 5B illustrates the adhesive backing 500 comprising the adhesive portion 503 and four releasable locking devices 506a-d. Each of the releasable locking devices 506a-d can be configured to attach to an intravenous extension tube of a multi-port indwelling catheter line in a manner similar to the releasable locking device 406 described in FIG. 4A. For example, suppose a patient is receiving intravenous therapy from a substance delivery system using a four-lumen indwelling catheter. In such a case, the patient can receive an injection at the injection site, and four lines can be protruding from the injection. An intravenous extension tube 300 (FIG. 3) can be displaced in between a portion of each of the four lines in proximity to the injection site. Specifically, each of the four releasable locking devices 506a-d of the adhesive backing 500 can attach an attachable portion of each of the intravenous extension tubes connected at a corresponding one of the four lines protruding from the patient. The adhesive backing 500 can hold the intravenous extension tubes while being secured to the patient in proximity to the injection site. The line securing system reflected by this example functions to protect the patient from enduring any injury as a result of accidently dislodging any of the lines or tubing that can be connected to the patient.

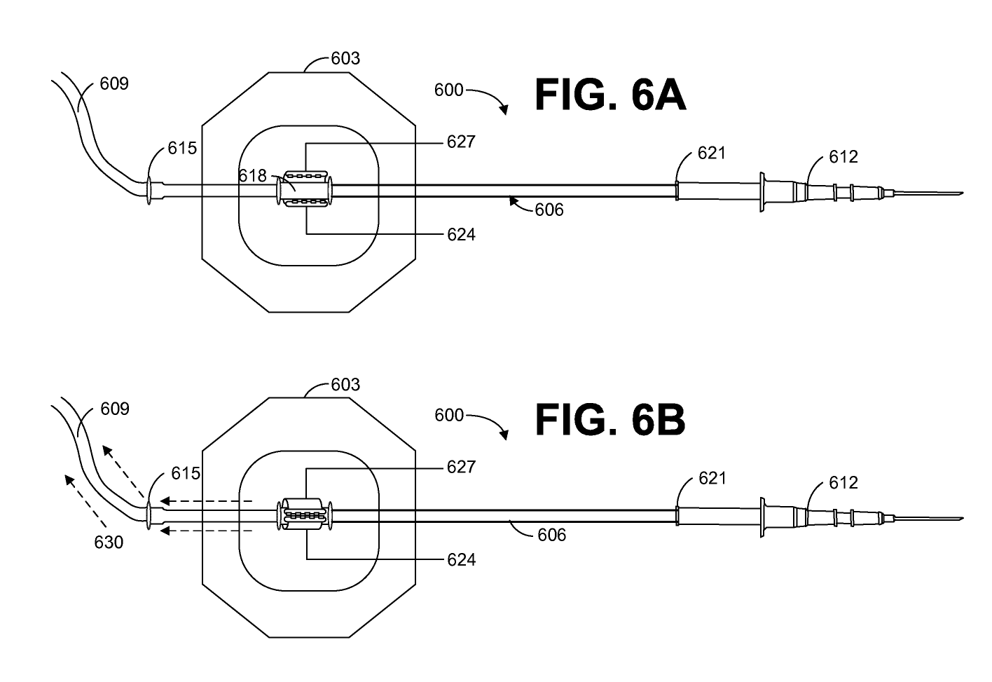

FIG. 6A illustrates an embodiment of an intravenous line securing system 600. The intravenous line securing system 600 comprises an adhesive backing 603, an intravenous extension tube 606, a source intravenous line 609, and an infusion member 612. Similar to the intravenous extension tube 300 described in FIG. 3, the intravenous extension tube 606 can comprise an inlet connector 615, an attachable portion 618, and an outlet connector 621. The source intravenous line 609 can be coupled to the inlet connector 615. The infusion member 612 can be coupled to the outlet connector 621.

The adhesive backing 603 comprises at least one releasable locking device 406 (FIGS. 4A-B) according to various embodiments. The releasable locking device 406 can comprise a lower semicircular arm 624 and an upper semicircular arm 627, wherein the upper semicircular arm 627 and the lower semicircular arm 624 are hingedly interconnected at one end and having interlocking portions at the other end. An aperture located in between the lower semicircular arm 624 and the upper semicircular arm 627 can be employed to detachably attach to the attachable portion 618 of the intravenous extension tube 606. The adhesive backing 603 can be configured to secure to a patient in proximity to and/or on the injection site. The source intravenous line 609 can be configured to channel solutions, or other content, to be directed to the patient at the injection site.

FIG. 6B illustrates one example of the functionality of the intravenous line securing system 600. Similar to the embodiment of the system described in FIG. 6A, The intravenous line securing system 600 comprises an adhesive backing 603, an intravenous extension tube 606, a source intravenous line 609, and an infusion member 612. Similar to the intravenous extension tube 300 described in FIG. 3, intravenous extension tube 606 can comprise an inlet connector 615 and an outlet connector 621, wherein the source intravenous line 609 is configured to couple to the inlet connector 615. The infusion member 612 can be coupled to the outlet connector 621.

As above in FIG. 6A, the adhesive backing 603 comprises at least one releasable locking device 406 (FIGS. 4A-B). The releasable locking device 406 can be structured similar to the releasable locking device 406 described in FIG. 6A. As can be appreciated, the releasable locking device 406 can be structured in any way such that the releasable locking device 406 can lock and unlock to the attachable portion of the intravenous extension tube 606. Further descriptions of embodiments of the releasable locking device 406 are described below in relation to FIG. 9.

Next, a description of the operation of various components of the intravenous line securing system 600 is provided. In one embodiment, if a force 630 is exerted upon the intravenous extension tube 606 and/or the source intravenous line 609, then the releasable locking device can be configured to detach from the attachable portion of the intravenous extension tube 606 to prevent the infusion member 612 from dislodging in the patient. The line securing system 600 can increase dwell times and decrease complications in the patient. For example, suppose a patient yawns and accidentally nudges or knocks down the source intravenous line. In this embodiment, the releasable locking device is configured to detach and thereby prevent the needle from dislodging and injuring the patient.

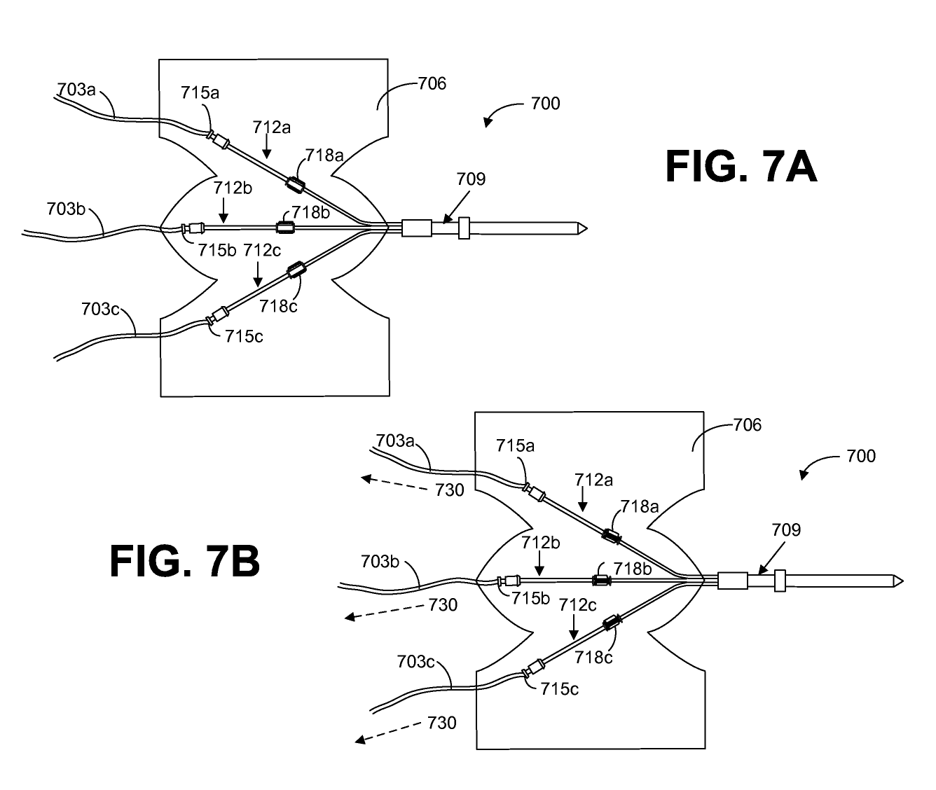

FIG. 7A illustrates an embodiment of a multi-port line securing system 700 comprising three lines 703a-c, an adhesive backing 706, and infusion member 709. The lines 703a-c can be intravenous lines or electric lines configured to monitor equipment or obtain cardiovascular readings, or other readings of the patient. The patient can be a human, an animal, any living being, or an electrical device, including but not limited to, a robot, vehicle, machine, or computer. The adhesive backing 706 is configured to attach to the patient. The infusion member 709 is configured to inject solutions into the patient's veins.

The adhesive backing comprises three releasable locking devices 506 (FIGS. 5A-B), each configured to detachably attach to an attachable portion of an intravenous extension tube 712a-c. Each of the three lines 703a-c respectively couple to three ports 715a-c. Each of the three ports 715a-c can be attached to one end of each intravenous extension tube 712a-c. The other end of the intravenous extension tube 712a-c can be attached to a portion of the infusion member 709. The releasable locking device 718a-c can operate similar to the releasable locking devices described in FIGS. 6A-B.

FIG. 7B illustrates one example of the functionality of the multi-port line securing system 700. Similar to the embodiment of the system described in FIG. 6A, the multi-port line securing system 700 comprises three lines 703a-c, an adhesive backing 706, and infusion member 709. The multi-port line securing system 700 is structured similar to the multi-port line securing system 700 described in FIG. 7A.

Next, a description of the operation of various components of the multi-port line securing system 700 is provided. In one embodiment, if a force 730 is exerted upon the intravenous extension tube 712a-c and/or the source intravenous line 703a-c, then the releasable locking devices 718a-c can be configured to detach from the attachable portion of the intravenous extension tube 712a-c to prevent the infusion member 709 from dislodging in the patient.

In one embodiment, the multi-port line securing system 700 can be configured such that only the respective releasable locking device 718a-c securing the dislodged source intravenous line 703a-c can be released. Alternatively, the multi-port line securing system 700 can be configured such that all the releasable locking devices 718a-c on the adhesive back 706 can be detached if one or more of the lines 703a-c and/or one or more of the intravenous extension tubes 712a-c have been dislodged from its original position.

According to some embodiments, the line securing system 600 (FIGS. 6A-6B), and the multi-port line securing system 700 can be pre-configured to respond to a specific pre-determined force. For example, suppose the line securing system 600 is built to secure an arterial line. In such a case, the line securing system 600 can be pre-configured to automatically detach at a specific amount of force that the line securing system 600 recognizes as creating a risk of dislodging the arterial line from the patient. As another illustrative example, suppose the multi-port line securing system 700 is built to secure a triple port central venous catheter. In such a case, a small amount of force can be enough to cause a risk of dislodgment or infection in the patient because the catheter can be inserted at sensitive points at the chest or neck. The multi-port line securing system 700 can be pre-configured to automatically detach at a minute amount of force that the multi-port line securing system 700 recognizes as creating a risk to the patient. Depending on the type of access device 315 (FIG. 3) inserted into the patient, the line securing system 600 or the multi-port line securing system 700 can require different amounts of force the detach the releasable locking device from the attachable portion of the intravenous extension tube.

A pre-defined force can be determined for each releasable locking device of the line securing system 600 or the multi-port line securing system 700. The pre-defined force measures the amount of force required to detach the releasable locking device from the intravenous extension tube. The pre-defined force can be measured by conducting experiments using intravenous therapy devices or drainage devices. The experiments can determine how much force is necessary to dislodge the device from the patient and/or how much force is necessary to injure the patient. The pre-defined force can be any amount of force that signals the releasable locking device to detach from the intravenous extension tube to prevent harm to the patient. As described above, different intravenous or drainage treatments require different amounts of force to trigger the detaching of the releasable locking device. As such, each releasable locking device can be manufactured based on the pre-defined force for a specific type of treatment.

According to various embodiments, the line securing system 600 and the multi-port line securing system 700 can be configured to have an adjustable amount of pre-defined force that is configurable, for example, by a nurse or other medical practitioner. For example, the medical practitioner can adjust the strength of the releasable locking device depending on the insertion site of the patient. Suppose the medical practitioner is using the line securing system 600 for an arterial line therapy by inserting a needle into an artery in the chest of the patient. The medical practitioner can recognize the fatal danger associated with any slight dislodgment of the arterial needle in the patient's chest. Then the medical practitioner can adjust the strength of the releasable locking device down such that the releasable locking device detaches upon the exertion of a small amount of force anywhere on the line securing system 600.

FIGS. 8A-8C shows perspective views of one embodiment of the releasable locking device 800. More specifically, FIG. 8A shows an embodiment of a locked releasable locking device 800. The releasable locking device 800 can comprise a lower semicircular arm 803 and an upper semicircular arm 806, wherein the upper semicircular arm 806 and the lower semicircular arm 803 are hingedly interconnected at one end and having interlocking portions 809 at the other end. An aperture 811 in between the lower semicircular arm 803 and the upper semicircular arm 806 can be employed to detachably attach to the attachable portion of the intravenous extension tube upon exertion of a pre-defined configurable force on the line and a latch 815 to further secure the interlocking portions 809.

FIG. 8B shows an embodiment of an unlocked releasable locking device 800. In this embodiment, the releasable locking device 800 comprises the same upper semicircular arm 806, lower semicircular arm 803, first interlocking portion 807, second interlocking portion 812, and latch 815. This releasable locking device 800 is structured similar to the one described in FIG. 8A. In one embodiment, an intravenous extension tube can rest upon the lower semicircular arm 803 and be attached to the releasable locking device 800 by lowering the upper semicircular arm 806 and interlocking the first interlocking portion 807 to the second interlocking portion 812. Finally, the intravenous extension tube can be further locked into the releasable locking device 800 by securing the latch 815.

FIG. 8C shows a top view of the releasable locking device shown in FIGS. 8A and 8B. As illustrated in FIG. 8C, the releasable locking device 800 top view comprises the latch 815 to the left, the interlocking portion 809 in the middle, and the upper semicircular arm 806 to the right.

FIGS. 9A-9F show alternative embodiments of the releasable locking device. In one embodiment, the releasable locking device 900 of FIG. 9A can comprise a base 903 configured to attach to an adhesive backing. The base 903 can comprise a holder 906 configured to receive and secure the attachable portion of the intravenous extension tube. The holder 906 can also be configured to detach the intravenous extension tube when a force is applied to the intravenous extension tube and/or a source intravenous line connected to the intravenous extension tube.

The releasable locking device 910 of FIG. 9B can comprise at least one lock 913 and an aperture 916. The releasable locking device 910 can be coupled to an adhesive backing. The aperture 916 can receive an intravenous extension tube. The lock 913 can further secure the intravenous extension tube to the releasable locking device 910. The releasable locking device 910 can be configured to release the intravenous extension tube when a force is applied to the intravenous extension tube and/or a source intravenous line connected to the intravenous extension tube.

FIG. 9C illustrates a releasable locking device 920 comprising a base 923, an aperture 926, and locks 929a-b. The releasable locking device 920 operates similar to the releasable locking devices 900 and 910 described in FIGS. 9A and 9B respectively.

FIG. 9D illustrates a releasable locking device 930 comprising a locking mechanism 933 and an aperture 936. The releasable locking device 930 operates similar to the other releasable locking devices described in FIGS. 9A-C.

FIG. 9E illustrates a releasable locking device 940 comprising at least two apertures 943a-b. The intravenous extension tube passes through both apertures 943a and 943b. The releasable locking device 940 is configured to lock the intravenous extension tube by attaching the bottom portion 946 and the top portion 949 of the releasable locking device 940. The releasable locking device 940 operates similar to the other releasable locking devices described in FIGS. 9A-D.

FIG. 9F illustrates a releasable locking device 950 comprising a base 953, holder 956, and aperture 959. The base 953 comprises at least two locks configured to attach to an adhesive backing to be secured to a patient. The holder 956 is configured to receive an intravenous extension tube through the aperture 959. The holder 956 is also configured to detachably attach to the intravenous extension tube similar to the embodiments described in FIGS. 9A-9E.

FIG. 9G illustrates a releasable locking device 960 comprising an aperture 963, adjustable knob 965, arms 966a-b, and a locking mechanism 969. The locking mechanism 969 can be a spring lock mechanism, a snap lock mechanism, or any other mechanism that holds the arms 966a-b together and allows for the arms to detachably attach to an intravenous extension tube when a force is exerted on the intravenous extension tube. The arms 966a-b can be pushed together to activate the locking mechanism 969 to hold an intravenous extension tube using the aperture 963. In addition, a user of the releasable locking device 960 can manually detach the releasable locking device 960 from the intravenous extension tube by pushing together the arms 966a-b and releasing the locking mechanism 969. The adjustable knob 965 can be used to adjust the strength of the releasable locking device 960. For example, if a medical practitioner is preparing to insert a drain into a nephrostomy insertion point on a patient's back, the medical practitioner can adjust the level of force necessary to release the releasable locking device 960 from the intravenous extension tube using the adjustable knob 965. The medical practitioner can also make such an adjustment after considering numerous other factors, including the patient's age, the patient's sensitivity, the insertion point, the sophistication of drain equipment used, other stabilization devices used, and/or any other consideration that can assist in determining the force necessary to detach the releasable locking device 960 from the intravenous extension tube.

FIG. 9H illustrates a releasable locking device 970 comprising upper arms 973 and a base 976. Upper arms 973 can comprise locks 981a. The base 976 can comprise a holder 979 and locks 981b. An intravenous extension tube can be positioned in the holder 979 and secured by snap locking into the locks 981b. A user of the releasable locking device 970 can secure an intravenous extension tube by pushing down the upper arms 973 and snapping the locks 981a into the locks 981b. As illustrated in FIG. 9H, the releasable locking device 970 can comprise other snap lock portions that can operate to connect the upper arm 973 to the base 976 and the intravenous extension tube. As can be appreciated, an attachable portion of the intravenous extension tube can detachably attach to any portion of the base 976 and be secured by locking the upper arm 973 onto the base 976. The upper arms 973 can release the locks 981a-b and detach the intravenous extension tube when a force is applied to the intravenous extension tube.

As can be appreciated, the releasable locking device can be made of plastic, metal, or any material sufficient to detachably attach to an intravenous line or an electric cable. The intravenous extension tube can be made of plastic, metal, or any material sufficient to secure to an intravenous tube and/or an access device. The force exerted on the one or more lines of the substance delivery system can be an unexpected accidental force or can be a pre-defined configurable force that is scheduled to dislodge an infusion member of an access device from the patient. The releasable locking device can be automatically detached or manually detached from the intravenous extension tube.

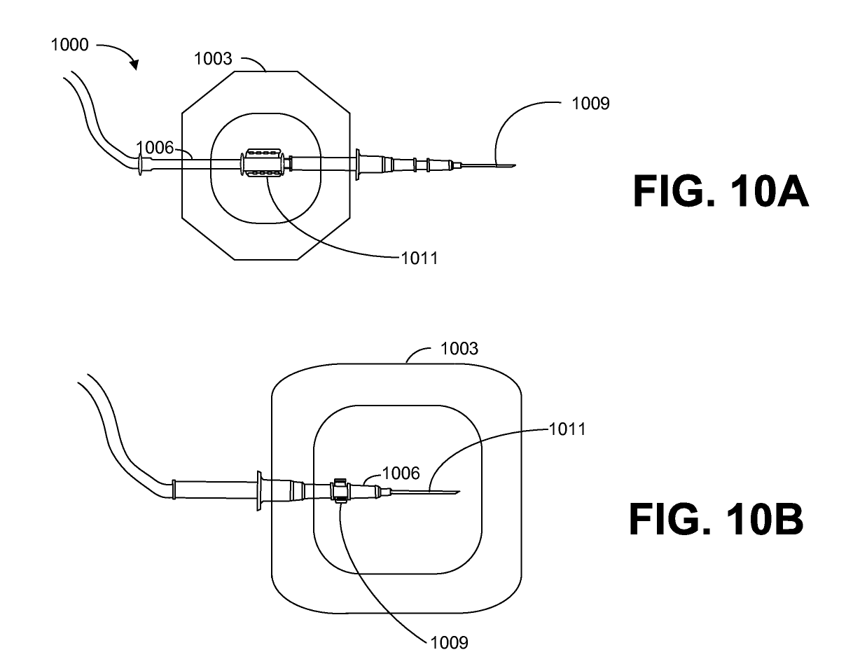

FIGS. 10A-B are drawings showing alternative embodiments of the intravenous line securing system depicted in FIGS. 6A-6B. FIG. 10A comprises the intravenous line securing system 1000 with at least one adhesive backing 1003, an intravenous extension tube 1006, and a needle 1011. The needle 1011 can alternatively be an insertion point of a drainage tube, electrical line, or any other insertion point of a line inserted into a patient's body. Adhesive backing 1003 comprises the releasable locking device 1009. As illustrated in FIG. 10A, the intravenous line securing system 1000 is secured onto the patient using the at least one adhesive backing 1003, a wrap, or other similar component. Adhesive backing 1003a secures the intravenous extension tube 1006 using the releasable locking device 1009. In addition, another adhesive backing 1003 can be used to secure the needle 1011 to the patient. If a pre-defined force is exerted on the intravenous line securing system 1000, the releasable locking device 1009 detaches from the intravenous extension tube 1006, but the needle 1011 and the adhesive backing 1003 can remain in place at the insertion site of the patient. The automatic detaching of the releasable locking device 1009 serves to prevent the needle 1011 from dislodging and injuring the patient.

FIG. 10B illustrates the embodiment of the intravenous line securing system 1000 using only one adhesive backing 1003 to secure the intravenous extension tube 1006 and the needle 1011. The adhesive backing 1003 comprises at least one releasable locking device 1009 configured to detachably attach to an attachable portion of the intravenous extension tube 1006. As illustrated in FIG. 10B, the intravenous extension tube 1006 is positioned closer to the needle 1011, allowing for the use of one adhesive backing 1003 to secure both the intravenous extension tube 1006 and the needle 1011. The releasable locking device 1009 operates to automatically detach from the intravenous extension tube 1006 when a pre-defined force is exerted upon the intravenous line securing system 1000.

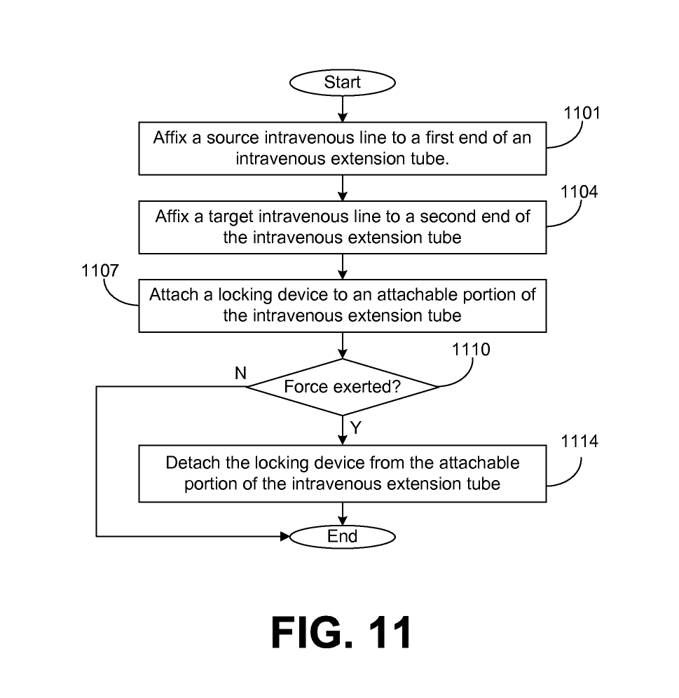

FIG. 11 is a flowchart illustrating one example of functionality implemented by a user of the substance delivery system comprising the line securing system according to various embodiments of the present disclosure. It is understood that the flowchart of FIG. 11 provides merely an example of many different types of functional arrangements that can be employed to implement the operation of the portion of the securing device as described herein. As an alternative, the flowchart of FIG. 11 can be viewed as depicting an example of steps and methods implanted by a patient or a doctor according to one or more embodiments.

Beginning with 1101, the user of the line securing system, such as a medical practitioner, affixes a source intravenous line to a first end of an intravenous extension tube. The source intravenous line can channel solutions from a substance delivery bag to be directed toward the veins of a patient. Alternatively, the source intravenous line can be an electric cable used to monitor equipment or vital patient information, including, but not limited to, cardiovascular readings. In 1104, the user of the line securing system affixes a target intravenous line to a second end of the intravenous extension tube. The target intravenous line can be a portion of an intravenous tubing that channels solutions toward an injection site of the patient. Alternatively, the target intravenous line can be an access device, such as the ones described above in relation to FIGS. 2A and 2B. Alternatively the target intravenous line can also be an electric cable used to monitor equipment and/or patient information.

In 1107, the user of the securing device system can attach the releasable locking device of an adhesive backing to an attachable portion of the intravenous extension tube. In 1110, if a force is exerted on the intravenous extension tube, the source intravenous line, or the target intravenous line, the releasable locking device detaches the locking device from the attachable portion of the intravenous extension tube, as depicted in 1114, after which the process ends. Alternatively, as shown in 1110, if a force is not exerted on the intravenous extension tube, the source intravenous line, or the target intravenous line, then the releasable locking device remains attached to the attachable portion of the intravenous extension tube, after which the process ends.

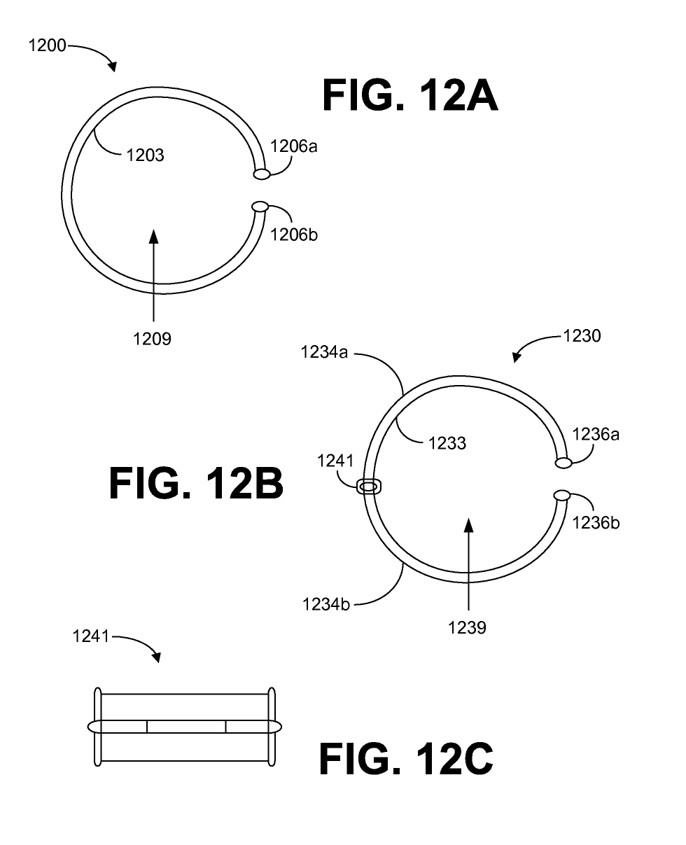

FIGS. 12A-C are drawings showing perspective views of various embodiments of an adhesive clamp 1200. The clamp 1200 can be structured, for example, as a C-clamp. The clamp 1200 comprises an interior surface 1203, two locking members 1206a and 1206b, and aperture 1209. The clamp 1200 can be made from a variety of plastic or foam materials, so long as it is flexible or resilient enough to detachably attach to the intravenous extension tube. As can be appreciated, the clamp 1200 can be any diameter such that the associated intravenous extension tube properly fits into the aperture 1209. Alternatively, the clamp 1200 can be made to have an adjustable diameter to accommodate a range of intravenous extension tube sizes.

In one embodiment, a medical practitioner can push the intravenous extension tube through the gap between the two locking members 1206a and 1206b. In such an embodiment, the flexible clamp 1200 will snap around the attachable portion of the intravenous extension tube. The medical practitioner can then lock the clamp 1200 by snapping the two locking members 1206a and 1206b together.

In some embodiments, the interior surface 1203 can comprise an adhesive layer to further secure the clamp 1200 to the intravenous extension tube. In such embodiments, the adhesive on the interior surface 1203 can adhere to the intravenous extension tube to further secure the intravenous extension tube in place to prevent dislodging any portion of the access device in the patient. In some embodiments, the adhesive on the interior surface 1203 can also be configured to detach from the attachable portion of the intravenous extension tube when a pre-defined force is exerted anywhere on the line securing system.

FIG. 12B shows an embodiment of a clamp 1230 comprising an interior surface 1233, two arms 1234a and 1234b, two locking members 1236a and 1236b, aperture 1239, and hinge 1241. Clamp 1230 is structured such that the two arms 1234a and 1234b are hingedly interconnected at hinge 1241. As can be appreciated, clamp 1230 can be made of any material that functions to attach and secure the intravenous extension tube, including, but not limited to, metal, plastic, foam, and/or rubber.

Clamp 1230 operates similar to clamp 1200, such that a medical practitioner can place the intravenous extension tube in the aperture 1239 between arms 1234a and 1234b and attach the two lock members 1236a and 1236b together around the attachable portion of the intravenous extension tube. Similar to the interior surface 1203 of clamp 1200, the interior surface 1233 can comprise an adhesive layer to further secure the clamp 1230 to the intravenous extension tube.

FIG. 12C is a drawing of an embodiment of hinge 1241. The arms 1234a and 1234b can be hingedly interconnected at hinge 1241 such that each arm 1234a and/or 1234b can move freely about the hinge.

In some embodiments, clamp 1200 and clamp 1230 can be separate and distinct from the releasable locking device in the line securing system. In such embodiments, the clamp 1200 or 1230 can first attach adhesively to the intravenous extension tube and then attach to the inside surface of the releasable locking device, serving to further prevent the infusion member and/or intravenous extension tube from dislodging in response to even the slightest movement occurring anywhere on the line securing system. The releasable locking device and the associated clamp can both be configured to detach from the intravenous extension tube when a pre-defined force is exerted anywhere upon the line securing system.

For example, if a force is exerted anywhere upon the intravenous extension and/or a source intravenous line, then the releasable locking device and the adhesive clamp can be configured to detach from the attachable portion of the intravenous extension tube to prevent the infusion member from dislodging in the patient. In one embodiment, the releasable locking device can be attached to the clamp such that the force required detach the releasable locking device can be enough to also detach the adhesive clamp from the attachable portion of the intravenous extension tube. In another embodiment, the adhesive clamp can be individually configured to release upon respectively different pre-determined amounts of force.

In an alternative embodiment, the releasable locking device in the line securing system can comprise an adhesive lining in the interior surface of the releasable locking device that directly attaches to the intravenous extension tube. In other words, the clamp 1200 or 1230 can be the releasable locking device itself. In such an embodiment, the adhesive layer of the releasable locking device further secures the intravenous extension tube. Additionally, the releasable locking device with the adhesive layer is configured to detach from the intravenous extension tube when a pre-defined force is exerted anywhere upon the line securing system.

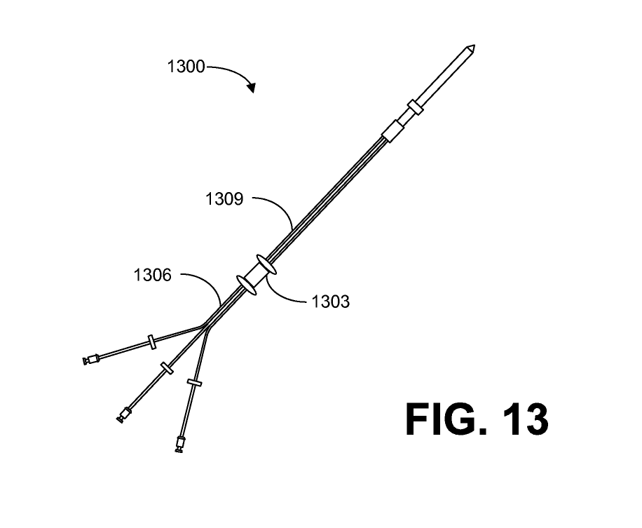

FIG. 13 is a drawing of an embodiment of an access device with a hub 1303 located on a catheter tube of the access device. Specifically, FIG. 13 shows an embodiment of a trilumen indwelling catheter 1300, similar to the multi-port indwelling catheter 250 shown in FIG. 2B. In one embodiment, the trilumen indwelling catheter 1300 can comprise a hub 1303 which is positioned in between the distal catheter tube 1306 and the proximal catheter tube 1309. In such an embodiment, the distal catheter tube 1306 is farther away from the infusion member of the access device than the proximal catheter tube 1309. In another embodiment, the hub 1303 can be positioned between the distal catheter tube 1306 and the intravenous extension tube. In another embodiment, the hub 1303 can be positioned between the intravenous extension tube and the proximal catheter tube 1309.

In one embodiment, the distal catheter tube 1306 can couple to another trilumen hub that has three openings at one end and one opening at the other end. The three openings of the trilumen hub can connect to three lines. The opening on the other side of the trilumen hub can connect to the distal catheter tube 1306. In another embodiment, the distal catheter tube 1306 can connect directly to any number of lines without the need for a second hub. In such an embodiment, the distal catheter tube 1306 can be manufactured such that the distal catheter tube 1306 divides into the necessary amount of separate catheter tubes, each separate catheter tube having a lumen port configured to connect directly to the line. For example, distal catheter tube 1306 can divide into three separate catheter tubes each of which can be configured to connect to one of the three lumens used in the trilumen indwelling catheter 1300.

The hub 1303 can comprise two openings; one opening can couple to the distal catheter tube 1306 and the other opening can couple to the proximal catheter tube 1306. The hub 1303 can act as a passageway for sealed fluid communication from the lumens through distal catheter tube 1306, through the hub 1303, through the proximal catheter tube 1309, and then through the infusion member into the patient. Alternatively, if the access device is a drainage tube, for example, then the hub 1303 acts as a passageway for sealed fluid communication in the reverse direction. As can be appreciated, the hub 1303 can also act as a passageway for an electrical line or any such line that can need to be connected to a patient, for example, to monitor the patient.

In some embodiments, the hub 1303 can comprise a pair of wings that can be secured to the skin of the patient by taping or by suturing. Alternatively, the placement of the hub 1303 can be such that the hub 1303 need not be secured to the skin of the patient. The use of the hub 1303 that is disposed in the catheter tubing can prevent catheter-related infections because the hub 1303 need not necessarily be sutured to the patient, thereby preventing possible infections and injuries related to the dislodgement of the hub 1303 and/or infusion member. Furthermore, the hub 1303 can be manufactured to be easier to disinfect and clean periodically, reducing the chance of infection in the hub 1303.

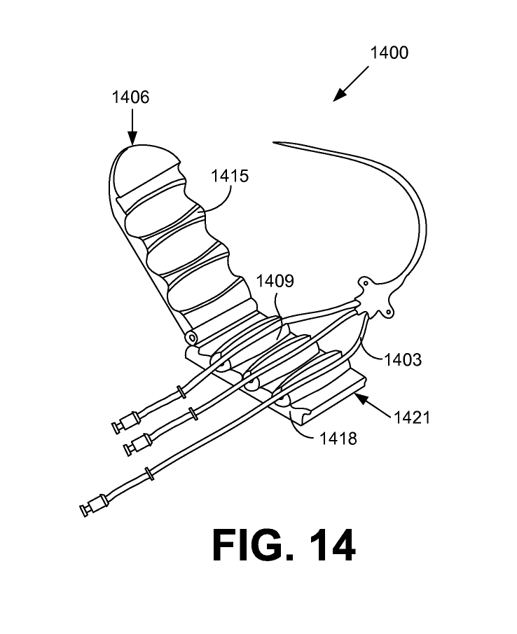

FIG. 14 is a drawing an intravenous line securing system 1400 according to various embodiments of the present disclosure. In the non-limiting example of FIG. 14, the intravenous line securing system 1400 comprises one or more intravenous lines 1403, a securing frame 1406, one or more line couplings 1409, and/or potentially other components. According to some embodiments, the securing frame 1406 can comprise an upper arm 1415, a lower arm 1418, a base 1421, and/or potentially other components.

In various embodiments, the line couplings 1409 are configured to receive and secure the intravenous lines 1403, for example, between the upper arm 1415 and the lower arm 1418. For example, an operator of the intravenous line securing system 1400, such as a medical professional, can insert an intravenous line 1403 into a line coupling 1409. In various embodiments, the line coupling 1409 is configured to at least partially surround and conform to the intravenous line 1403 tightly, such that the intravenous line 1403 has restricted movement relative to the line coupling 1409. For example, once the line coupling 1409 has at least partially surrounded at least a portion of the intravenous line 1403, the line coupling 1409 will not be able to slide or move relative to the intravenous line 1403.

The line couplings 1409 can detachably attach to a portion of the lower arm 1418. In various embodiments, the line couples 1409 are configured to snap into recesses in the upper arm 1415 and/or the lower arm 1418. As shown in FIG. 14, the upper arm 1415 and the lower arm 1418 of the securing frame 1406 are hingedly interconnected at one end, such that the upper arm 1415 and the lower arm 1418 can swivel around the hinged interconnection. According to some embodiments, a user of the intravenous line securing system can close the securing frame 1406 by latching down a portion of the upper arm 1415 to a portion of the lower arm 1418. When the securing frame 1406 is latched together, a portion of the upper arm 1415 and/or the lower arm 1418 can tightly attach to at least a portion of the line coupling 1409. In this regard, latching the securing frame 1406 further facilitates securing the intravenous line 1403 between the upper arm 1415 and the lower arm 1418. The base 1421 of the securing frame 1406 is configured to detachably attach to a patient, for example, by attaching to the clothing of a patient or to a bandage of a patient.

The intravenous line securing system 1400 can be configured to facilitate an inserted point (not shown) of the intravenous lines 1403 from dislodging and injuring the patient. To this end, when the intravenous line securing system 1400 is attached to the patient, the stability and control provided by the securing frame 1406 and the line couplings 1409 prevent movement of the portion of the intravenous lines 1403 that are closer to the insertion points. Therefore, the prevention of movement of the intravenous lines 1403 relative to the insertion points prevent injury to the patient that can occur when a force is exerted at various locations on the intravenous line.

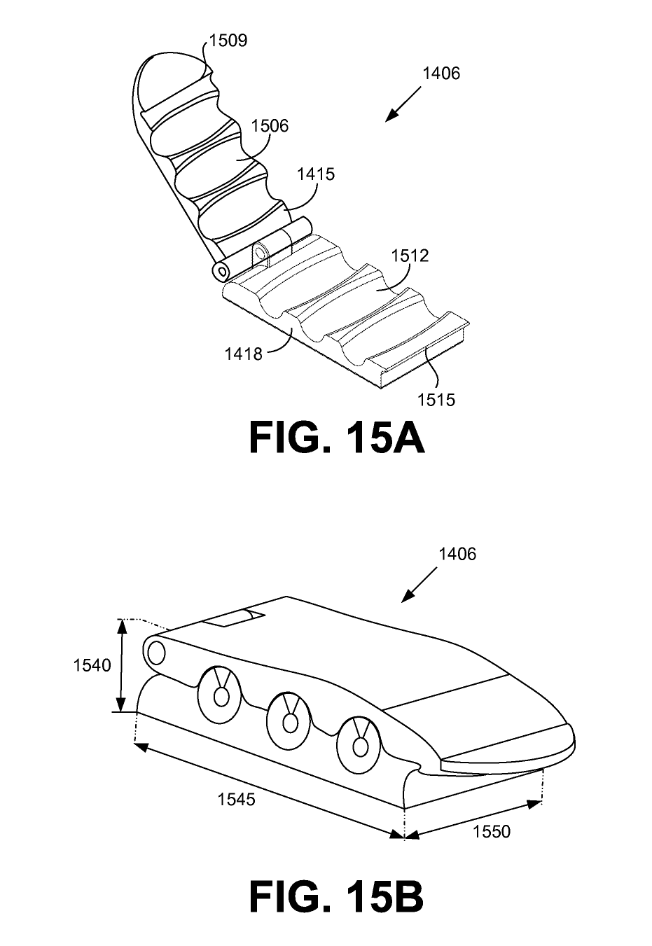

Referring now to FIG. 15A, shown is another drawing of the securing frame 1406 depicted in FIG. 14. Similar to the securing frame 1406 shown in FIG. 14, the securing frame 1406 comprises the upper arm 1415, the lower arm 1418, and the base 1421. In the embodiment shown in FIG. 15A, the securing frame further comprises a hinged connection 1503, upper concave portions 1506, an upper latch 1509, lower concave portions 1512, a lower latch 1515, and/or potentially other components. The hinged connection 1503 facilitates movement of the upper arm 1415 relative to the lower arm 1418. In one embodiment, the hinged connection 1503 can comprise a metal (e.g., steel) or plastic bearing, or a bearing made of any other suitable material, configured to stabilize the upper arm 1415 and the lower arm 1418 such that they can move relative to one another. For example, the upper arm 1415 may be configured to move about the hinged connection 1503 a predefined amount, such as 180 degrees, 90 degrees, 45 degrees, etc.

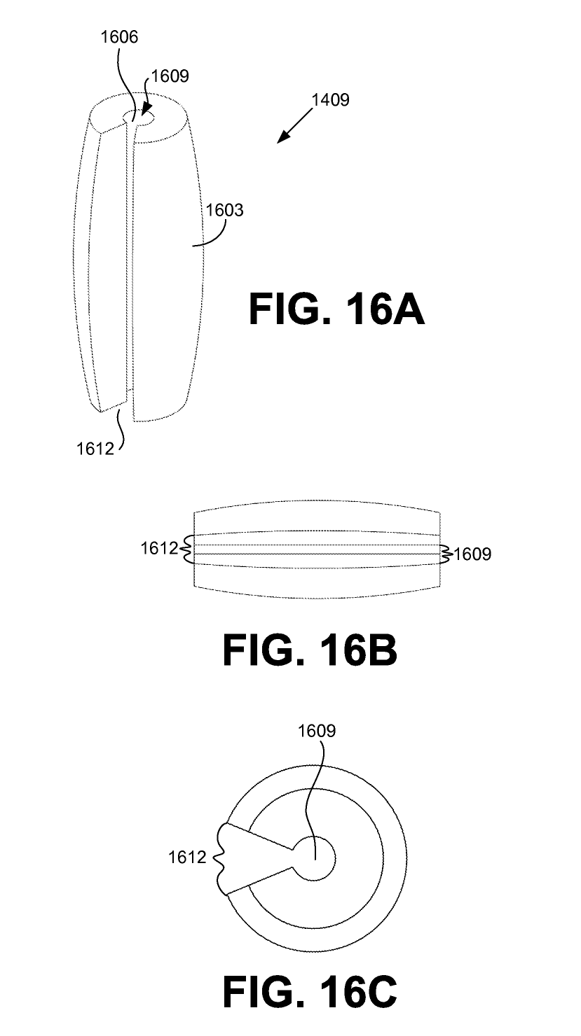

The upper concave portions 1506 and the lower concave portions 1512 facilitate receiving and securing the line couplings 1409. As will be further described below with reference to FIGS. 16A-C, an embodiment of the line coupling 1409 can be at least partially oblong and/or cylindrical. For this reason, the upper concave portions 1506 and the lower concave portions 1512 are shaped to accommodate the shape of the line coupling 1409 and tightly surround the line coupling 1409 when the securing frame 1406 is enclosed. It can be appreciated that the line couplings 1409 can be built in any shape. Therefore, it can also be appreciated that the upper concave portions 1506 and the lower concave portions 1512 can also be structured as necessary to tightly surround any shape of the line coupling 1409. The upper latch 1509 and the lower latch 1515 can interlock, or overlap, to secure the upper arm 1415 to the lower arm 1418 to restrict movement of the intravenous lines 1403.

Moving on to FIG. 15B, shown is a side view of the securing frame 1406 when the upper arm 1415 and the lower arm 1418 are latched. In various embodiments, the securing frame 1406, when closed, can be a height 1540 of about 8 cm to about 10 cm. In one embodiment, a length 1545 of the base 1421 of the securing frame 1406 can about 40 cm to about 50 cm. In various embodiments, a width 1510 of the base 1421 of the securing frame 1406 can be about 18 cm to about 20 cm. Other suitable dimensions may be appreciated.

The securing frame 1406 can be made of any material configured to attach to and secure the line couplings 1409. For example, the securing frame 1406 can be made of a plastic, such as a polycarbonate. In some embodiments, portions of the securing frame 1406 can be lined with a gripping material and/or an adhesive material to facilitate securing the line couplings 1409 and/or the intravenous lines 1403 directly to those portions of the securing frame 1406. The line couplings 1409 can be made of any material configured to at least partially enclose a portion of an intravenous line 1403. In some embodiments, the line coupling 1409 can be made of a partially flexible, but sturdy material, such as, for example, silicone or urethane.

Next, with respect to FIG. 16A, shown is another drawing of the line coupling 1409 depicted in FIG. 14. The embodiment of the line coupling shown in FIG. 16A can comprise an outer surface 1603, an inner surface 1606, an aperture 1609, a slit 1612, and/or potentially other components. As shown in FIG. 16A, the line coupling 1409 can be shaped at least partially cylindrical with tapered ends such that a diameter at the ends of the line coupling 1409 are smaller than a diameter in the center. The outer surface 1603 can be configured to detachably attach to a portion of the securing frame 1406. In one embodiment, the outer surface 1603 can comprise any material, such as, for example, an adhesive, that facilitates securing the line coupling 1409 to the securing frame 1406.

The inner surface 1606 can be configured to at least partially surround and enclose a portion of the intravenous line 1403 via the aperture 1609. In various embodiments, the aperture 1609 is substantially similar in shape and/or size to closely conform to an intravenous line such that the intravenous line fits tightly in the aperture 1609. For example, in various embodiments, the aperture 1609 can be the same diameter, slightly larger in diameter, or slightly smaller in diameter than a diameter of the intravenous line 1403 being secured. In one embodiment, the inner surface 1606 can comprise a material, such as, for example, an adhesive, to facilitate securing the intravenous line 1403 to the inner surface 1606. In one embodiment, the line coupling 1409 can be manufactured as part of the intravenous line 1403 itself.

In one embodiment, the line coupling 1409 can be only partially enclosed. In such an embodiment, the line coupling 1409 can comprise the slit 1612 along a side of the line coupling. The line coupling 1409 can be configured to receive the intravenous line 1403 through the slit 1612. In another embodiment, the line coupling 1409 can be fully enclosed such that a user can slide the intravenous line 1403 through the aperture 1609. In various embodiments, the slit 1612 may be temporarily opened (e.g., via a removable opening) to insert or remove an intravenous line within the aperture 1609.

In one embodiment, the line coupling 1409 has an inherent gripping strength that firmly secures the intravenous line 1403, while the line coupling 1409 retains the ability to slide up and down the intravenous line 1403. In this manner, a user can adjust the location of the line coupling 1409 easily without having to take the intravenous line 1403 out of the line coupling 1409. FIGS. 16B and 16C are cross sectional drawings of the line coupling 1409 depicted in FIG. 16A.

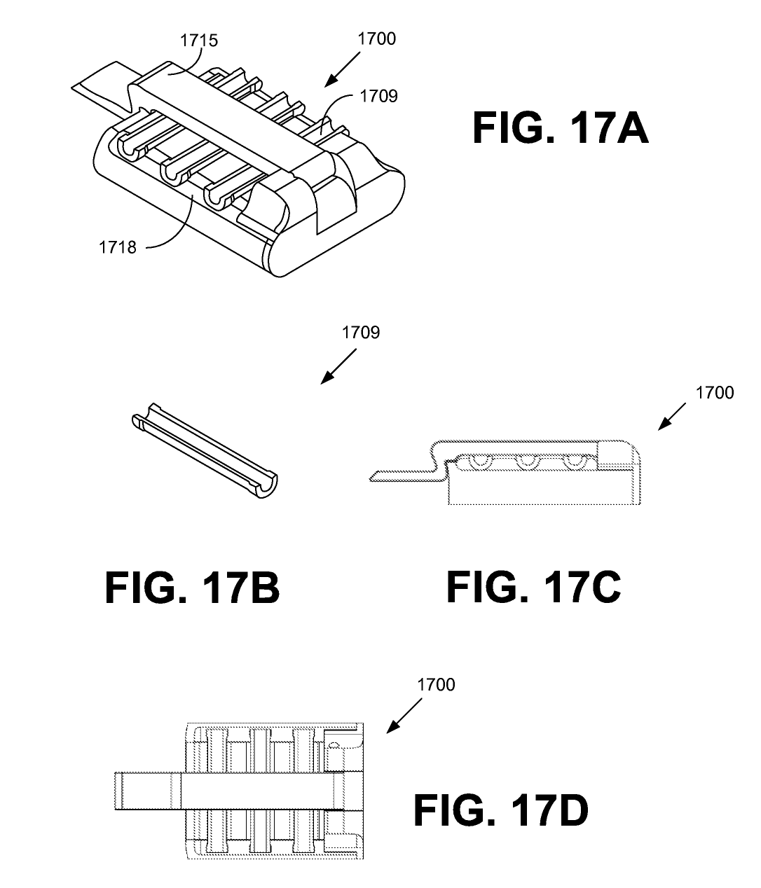

Moving on to FIG. 17A, shown is a perspective view of another embodiment of an intravenous line securing system 1700. As shown in the non-limiting example of FIG. 7A, the intravenous line securing system 1700 comprises a securing frame 1706, line couplings 1709, and/or other components. The securing frame 1706 can be structured similar to the securing frame 1406 described in FIG. 14. For example, the securing frame 1706 can comprise an upper arm 1715, lower arm 1718, and/or other components. In one embodiment, the upper arm 1715 can be of a smaller width relative to the width of the lower arm 1718, as shown in FIG. 17A.

The line couplings 1709 can be structured similar to the line couplings 1409 described above with respect to FIG. 14 and FIGS. 16A-C. For example, the line couplings 1709 of FIGS. 17A-D can be made of gripping material, such as silicone. The gripping material can facilitate securing the intravenous lines 1403 such that the intravenous line 1403 has restricted movement, if any movement at all, relative to the line coupling 1709.

As shown in FIG. 17B, the line coupling 1709 can comprise a concave portion 1740 configured to receive and secure an intravenous line 1403. In some embodiments, the line coupling 1709 can be configured to detachably attach to the securing frame 1706. As shown in FIG. 17B, the line coupling 1709 can be embodied as a semicircular tube comprising the gripping material. In this regard, the intravenous line 1403 can securely rest on top of the semicircular tubed line coupling 1709 such that the intravenous line 1403 cannot be moved relative to the line coupling 1709. FIGS. 17C and 17D show a side view and a top view, respectively, of the intravenous line securing system 1700 depicted in FIG. 17A.

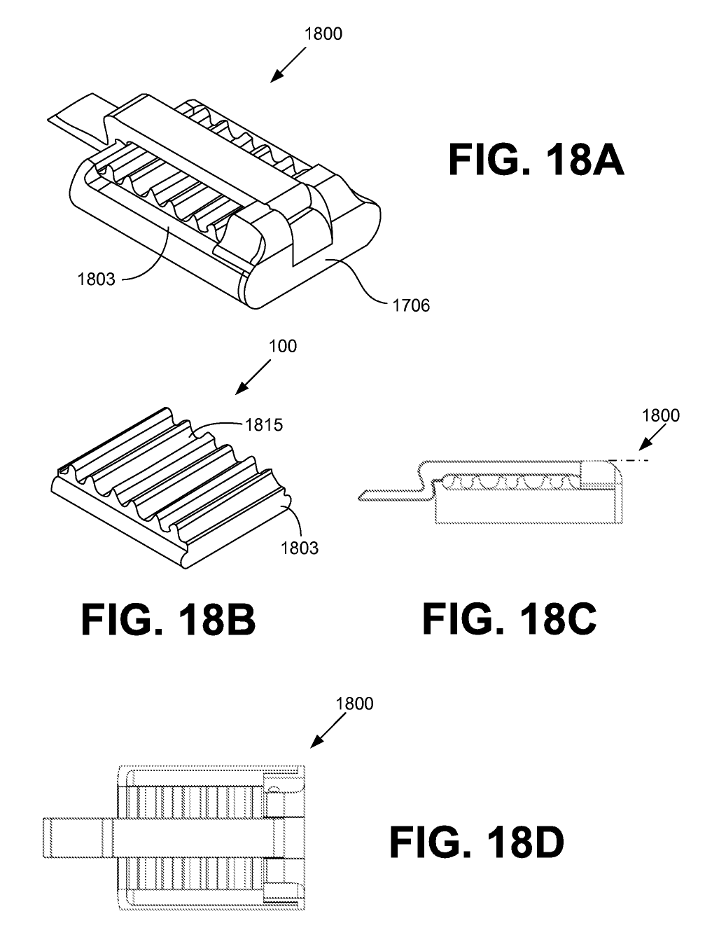

Referring next to FIG. 18A, shown is a perspective view of another embodiment of an intravenous line securing system 1800. In the non-limiting example of FIG. 18A, the intravenous line securing system 1800 comprises the securing frame 1706, a base 1803, and/or potentially other components. The base 1803 can function similar to the line couplings 1709 described with reference to FIG. 17 except that the base 1803, as opposed to a lower arm, can receive and secure a plurality of intravenous lines 1403. As shown in FIG. 18B, the base 1803 can comprise a plurality of concave portions 1815, each one configured to receive and secure an intravenous line 1403. Similar to the line couplings 1709 described in FIG. 17, the base 1803 can be made of a gripping material, such as, for example, silicone. The gripping material can be configured to prevent movement of the intravenous line 1403 relative to each concave portion 1815 of the base 1803. In one embodiment, the base 1803 can be detachably attached to the securing frame 1706. FIGS. 18C and 18D show a side view and a top view of the embodiment of the intravenous line securing system 1800 depicted in FIG. 18A, respectively.

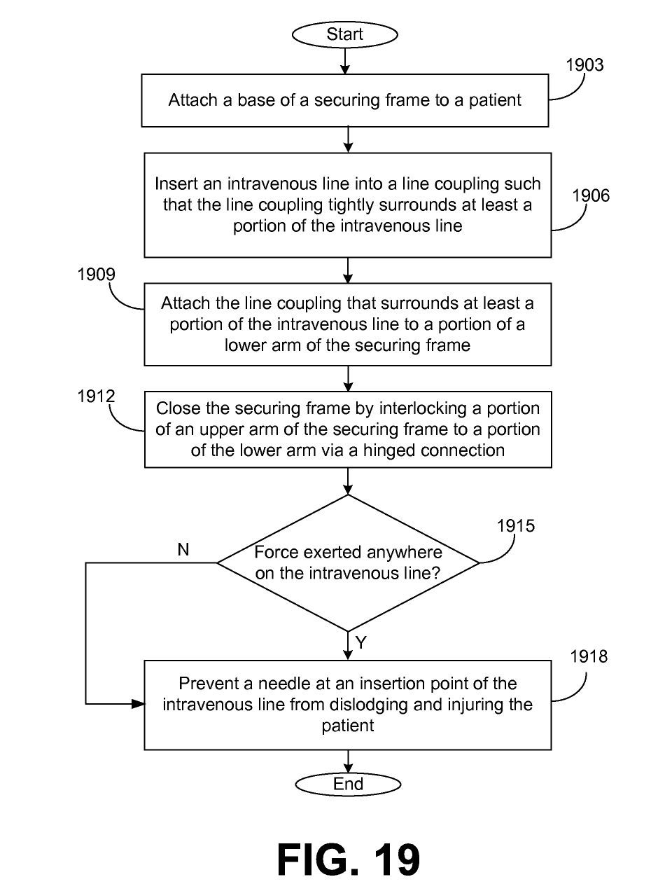

FIG. 19 is a flowchart illustrating one example of functionality implemented by a user of the substance delivery system 100 comprising the intravenous line securing system according to various embodiments of the present disclosure. It is understood that the flowchart of FIG. 19 provides merely an example of many different types of functional arrangements that can be employed to implement the operation of the portion of the securing device as described herein. As an alternative, the flowchart of FIG. 19 can be viewed as depicting an example of steps and methods implanted by a patient or a doctor according to one or more embodiments.

Beginning with 1903, the user of the intravenous line securing system 1400, such as a medical practitioner, attaches a base 1421 of the securing frame 1406 to a patient. For example, the base 1421 can comprise a structure that facilitates suturing the securing frame 1406 to the patient. As another illustrative example, the base 1421 can comprise an adhesive material, or a clip clipped to an adhesive material, strong enough to detachably attach the securing frame 1406 securing the intravenous lines 1403 using the line couplings 1409 to the patient. As yet another illustrative example, the base 1421 can be attached, for example, by adhesive, to an adhesive bandage that is detachably attachable to the patient.

In 1906, the user then inserts an intravenous line 1403 into the line coupling 1409, for example, via the slit 1612. Upon insertion of the intravenous line 1403 into the line coupling 1409, the line coupling 1409 can at least partially surround a portion of the intravenous line 1403. In one embodiment, the line coupling 1409 can comprise a material, such as an adhesive or silicone, that facilitates further securing the intravenous line 1403 to the line coupling 1409.

In 1909, the user can attach the line coupling 1409, which is surrounding and supporting at least a portion of the intravenous line 1403, to a portion of the lower arm 1418 of the securing frame 1406. For example, the user can attach the line coupling 1409 to the upper concave portion 1506 of the upper arm 1415 and/or the lower concave portion 1512 of the lower arm 1418. In 1912, the user can close and/or lock the securing frame 1406 by interlocking a latching mechanism on the securing frame 1406. For example, the user can snap together a portion of the upper arm 1415 onto and underneath a portion of the lower arm 1418 to secure the line couplings 1409 into a predefined location. Once the securing frame is latched, the intravenous lines 1403 are secured in the predefined locations such that when a force is exerted anywhere on the intravenous lines 1403, the insertion point of the intravenous line 1403 can remain in position. In this regard, in 1915, if a force is exerted anywhere on the intravenous line 1403, then, in 1915, the intravenous line securing device 1400 prevents a needle at the insertion point from dislodging and injuring the patient. To this end, the fastening of the securing frame 1406 tightly around flexible line couplings 1409 that at least partially surround and secure the intravenous line 1403 in proximity to the insertion point prevents injury to the patient.

As can be appreciated, the securing frame 1406 and 1706 can be structured in any manner such that a line coupling 1409 or an intravenous line 1403 can be received and secured using the securing frame 1406. In this regard, the securing frame 1406 can be structured similar to the releasable locking devices shown in FIGS. 8 and 9 and the clamp shown in FIG. 12. Similarly, the securing frame 1406 and 1706 can be positioned at any portion of the intravenous line 1403. For example, the securing frame 1406 and 1706 can be positioned closer to the insertion point or farther away from the insertion point.

It should be emphasized that the above-described embodiments of the present disclosure are merely possible examples of implementations set forth for a clear understanding of the principles of the disclosure. Many variations and modifications can be made to the above-described embodiment(s) without departing substantially from the spirit and principles of the disclosure. All such modifications and variations are intended to be included herein within the scope of this disclosure and protected by the following claims.

It should be noted that ratios, concentrations, amounts, and other numerical data can be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. In an embodiment, the term “about” can include traditional rounding according to significant figures of the numerical value. In addition, the phrase “about ‘x’ to ‘y’” includes “about ‘x’ to about ‘y.’”

Disjunctive language such as the phrase “at least one of X, Y, or Z,” unless specifically stated otherwise, is otherwise understood with the context as used in general to present that an item, term, etc., can be either X, Y, or Z, or any combination thereof (e.g., X, Y, and/or Z). Thus, such disjunctive language is not generally intended to, and should not, imply that certain embodiments require at least one of X, at least one of Y, or at least one of Z to each be present.

It should be emphasized that the above-described embodiments of the present disclosure are merely possible examples of implementations, and are merely set forth for a clear understanding of the principles of this disclosure. Many variations and modifications can be made to the above-described embodiment(s) of the disclosure without departing substantially from the spirit and principles of the disclosure. All such modifications and variations are intended to be included herein within the scope of this disclosure and protected by the following claims.

Therefore, the following is claimed:

1. A system, comprising:
a plurality of intravenous lines;
a securing frame comprising an upper arm, a lower arm, and a base, the upper arm and the lower arm being hingedly interconnected at one end of the securing frame and having interlocking portions at another end of the securing frame, the upper arm and the lower arm comprising a plurality of concave portions configured to receive and secure a plurality of line couplings between the upper arm and the lower arm, wherein the base of the securing frame is configured to detachably attach to an adhesive bandage securable to a patient; and
wherein each of the line couplings comprise an aperture configured to receive and secure at least one of the intravenous lines such that the at least one of the intravenous lines has restricted movement relative to each of the line couplings, and each of the line couplings being configured to detachably attach to at least one of the plurality of concave portions of the upper arm and the lower arm.

2. The system of claim 1, wherein the at least one intravenous line is configured to channel a liquid substance in an intravenous line bag.

3. The system of claim 1, wherein the at least one intravenous line couples to an access device.

4. The system of claim 3, wherein the access device is selected from a group consisting of a multi-port indwelling catheter, an arterial line, a central line, a peripherally inserted central catheter line, and a peripheral intravenous line.

5. A frame for securing at least one intravenous line, comprising:
an upper arm, a lower arm, and a base, the upper arm and the lower arm being hingedly interconnected at one end of the frame and having interlocking portions at another end of the frame, the upper arm and the lower arm comprising at least one portion configured to receive and secure at least one line coupling between the upper arm and the lower arm; and
wherein the at least one line coupling comprises an aperture configured to receive and secure the at least one intravenous line, and the line coupling being configured to detachably attach to at least one of the plurality of concave portions of the upper arm and the lower arm.

6. The frame of claim 5, wherein the base of the securing frame is configured to detachably attach to an adhesive bandage securable to a patient.

7. The system of claim 5, wherein the at least one line coupling further comprises a slit along a side of the at least one line coupling, the at least one line coupling being configured to receive the intravenous line through the slit.

8. The system of claim 5, wherein the at least one line coupling is configured to secure the at least one intravenous line by coupling the at least one intravenous line to an inner surface of the at least one coupling, wherein the at least one coupling is detachably attachable the securing frame.

9. The system of claim 5, wherein the at least one intravenous line is selected from a group consisting of a drainage tube, an electric line, and an intravenous tube.

10. The system of claim 5, wherein at a portion of the securing frame comprises silicone to facilitate preventing the at least one intravenous line from moving relative the securing frame.

11. The system of claim 5, wherein an inner surface of the at least one line coupling comprises an adhesive material configured to detachably attach to the intravenous line.

12. The system of claim 5, wherein a base of the securing frame is configured to detachably attach to a patient.

13. The system of claim 5, wherein at least one intravenous line has restricted movement when the upper arm and the lower arm are interlocked around the line coupling.

14. The system of claim 5, wherein the at least one portion of the securing frame is configured to receive and secure the at least one intravenous line.

15. A method for stabilizing an intravenous line, comprising:
attaching a base of a securing frame to a patient;
receiving at least a portion of an intravenous line through an aperture along a side of a line coupling, the line coupling configured to facilitate securing the at least a portion of the intravenous line to a patient;
attaching the line coupling onto a lower arm of securing frame;
interlocking a portion of an upper arm of the securing frame with a portion of the lower arm of the securing frame such that the line coupling is secured within the securing frame and the intravenous line is secured within the line coupling; and
preventing an insertion point of the intravenous line from dislodging in response to interlocking the portion of the upper arm of the securing frame with the portion of the lower arm of the securing frame.

16. The method of claim 15, wherein the line coupling further comprises an adhesive material on an inner surface of the line coupling, the adhesive material configured to facilitate securing the intravenous line to the patient.

17. The method of claim 15, wherein the upper arm and the lower arm further comprise at least one concave portion configured to receive the line coupling, the at least one concave portion on the upper arm and the lower arm configured to facilitate securing the intravenous line to the patient.

18. The method of claim 15, wherein line coupling comprises silicone.

19. The method of claim 15, wherein the securing frame comprises a polycarbonate material.

20. The method of claim 15, wherein the line coupling is affixed to the at least a portion of the intravenous line.
